(19) United States
(12) Patent Application Publication
(10) Pub. No.: US 2009/0324663 A1
(43) Pub. Date: Dec. 31, 2009

(54) KIT FOR FORMULATING A COSMETIC PRODUCT

(75) Inventors: Jean- Yves LEGENDRE, Paris (FR); Carole Guiramand, Jouy En Josas (FR); Guillaume Cassin, Villebon Sur Yvette (FR); Jean-Thierry Simonnet, Cachan (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/555,161

(22) Filed: Sep. 8, 2009

Related U.S. Application Data
(62) Division of application No. 11/105,528, filed on Apr. 14, 2005, now abandoned.
(60) Provisional application No. 60/567,312, filed on May 3, 2004.

(30) Foreign Application Priority Data
Apr. 19, 2004 (FR) 0404125

(51) Int. Cl. A61L 9/01; A61K 8/00; A61K 8/02; A61K 47/30; A61Q 13/00; A61Q 5/00

(52) U.S. Cl. 424/401; 424/59; 424/76.1; 514/772; 514/772.3; 514/773; 514/777; 424/65; 512/1

(57) ABSTRACT

The present invention relates to kits for formulating a cosmetic product, containing an aqueous composition and at least one water-soluble anhydrous film, the aqueous composition and the film(s) being mixed together extemporaneously to form the cosmetic product.

### REFERENCE TO PRIOR APPLICATIONS

This application claims priority to U.S. provisional application 60/567,312 filed May 3, 2004, and to French patent application 0404125 filed Apr. 19, 2004, both incorporated herein by reference.

### FIELD OF THE INVENTION

The present invention relates to kits for formulating cosmetic products, and in particular to a kit for formulating cosmetic products comprising at least one anhydrous thin film and an aqueous composition, the film and the composition being intended to be mixed together extemporaneously to form a cosmetic product, which may especially be a makeup or care product for the skin, mucous membranes, the integuments or the hair.

The invention also relates to the use of the so made product for the cosmetic treatment of the skin, mucous membranes, the integuments or the hair, and the so made product itself.

The invention also relates to a process for formulating the product using a kit.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Cosmetic products contain various compounds that each play a role, as regards either the presentation form of the product, the activity of the product or any other property of the product, for instance its odour or its colour. Thus, for example, the presence of active agents contributes towards the efficacy of the product on the skin, for example to keep the skin healthy and/or to improve the condition of the skin, while viscosity agents make it possible to adjust the texture of the product, and dyes impart a given shade thereto. In addition, usually, these products contain a large number of different compounds, and it may so happen that it is not desirable, for various reasons, for all of the compounds be mixed together before using the product. For example, compounds, and especially active agents, may be mutually incompatible or may cause instability of the composition comprising them.

Moreover, users are increasingly in search of a composition that is suitable for the use they wish to make of it as and when they are so inclined. Thus, according to mood, the season or external temperature, the user wishes to have available a composition that is suited to his or her need of the moment, for example to have available a composition that is more or less thickened or more or less concentrated in active material or that has a particular coloration or fragrance, these features possibly varying over time.

### SUMMARY OF THE INVENTION

The kit forming the subject of the present invention makes it possible to overcome these problems. This kit has several embodiments.

### DETAILED DESCRIPTION OF THE PREFERRED EMBODIMENTS

One subject of the invention is a kit for formulating a cosmetic product, comprising (1) an aqueous composition comprising at least one fatty substance, and (2) at least one water-soluble anhydrous film comprising at least one compound that is capable of modifying the aqueous composition.

This composition and the film(s) are mixed together extemporaneously to form the cosmetic product.

This cosmetic product may be used especially as a care or makeup product for the skin, mucous membranes, the integuments or the hair.

In the present patent application the expression “compound capable of modifying the aqueous composition” means any compound that will modify the activity and/or the appearance of the composition, for example its activity by the addition of one or more active agents, or its appearance by addition of a thickener (modification of the viscosity) or by addition of a dye (modification of the colour) or by addition of a fragrance (modification of the odour).

The term “fatty substance” means any water-insoluble compound, this compound possibly being, for example, an oil (liquid fatty substance) or a solid fatty substance. It is preferably an oil.

The extemporaneous mixture is obtained by simple mixing of the desired amounts of the aqueous composition and of the film(s), these amounts being determined according to the desired final aim. The mixture obtained constitutes a single use cosmetic product. However, the kit may contain several films of identical or different compositions, which makes it possible to obtain several times a cosmetic product that is identical or different at each use.

A subject of the invention is also a kit for formulating a cosmetic product, comprising:
i) an aqueous composition; and
ii) at least one water-soluble anhydrous film comprising at least one compound which, for reasons of stability, can only be mixed with the composition extemporaneously in order to form the cosmetic product.

A subject of the invention is also a kit for formulating a cosmetic product, comprising:
i) an aqueous composition;
ii) at least one first water-soluble anhydrous film; and
iii) at least one second water-soluble anhydrous film, the second film being different from the first, especially with regard to the concentration and/or the nature of at least one compound it contains.

The cosmetic product is obtained by adding one or more of the first films and one or more of the second films to the aqueous composition.

A subject of the invention is also a kit for the customized formulation of a cosmetic product, especially a care or makeup product, comprising
i) an aqueous composition;
ii) a plurality of identical or different water-soluble anhydrous films, intended to be mixed with the aqueous composition to form the cosmetic product, and
iii) instructions, especially on an explanatory note, for custom-formulating the cosmetic product as a function of the number of identical or different watersoluble anhydrous films to be mixed with the composition. The expression “customized formulation” is understood to mean a formulation adopted to the consumer's requirements at the moment of use.

A subject of the invention is also a process for formulating a cosmetic product, especially a care or makeup product, which comprises:
a) determining at least one characteristic of the cosmetic product to be formulated, especially a shade or a concentration, the characteristic being due to at least one compound present either in a plurality of identical water-soluble anhydrous films, or in a plurality of water-soluble anhydrous films that are different from each other, especially as regards the concentration and/or the nature of at least one compound they contain; and
b) adding to an aqueous composition a number of the identical or different films, the number depending on the characteristic of the cosmetic product to be formulated.

A subject of the invention is also a process for modifying the rheology, colour and/or fragrance characteristics of an aqueous composition for the purpose of formulating a cosmetic product, especially a care or makeup product, which comprises adding to an aqueous composition one or more water-soluble anhydrous films comprising one or more compounds capable of modifying the rheology, the colour and/or the fragrance of an aqueous composition.

It is known practice to use anhydrous films that dissolve immediately on contact with water or saliva, and edible films for wrapping foods in order to increase their shelf life, and this technique has been the subject of patent applications (for example U.S. Pat. Nos. 5,965,708, 5,962,053 and JP-A-10/215,792). Similarly, this type of technology is used in the pharmaceutical field to administer active principles orally in the form of formulations that break down instantaneously in the mouth (see, for example, documents WO-A-2002/085119, WO-A-2002/043657 and WO-A-2001/070194), or by application to other mucous membranes, for instance the vagina (EP-A-1 110 546) and to wounds (JP-A-63/220876). In addition, document JP-A-2002/212027 describes the production and composition of cosmetic preparations in the form of water-soluble films, and documents WO-A-2002/05789, US-A-2002/0127254 and WO-A-2003/075812 describe the production and administration of anhydrous polymeric films for a direct administration of cosmetic compositions to premoistened skin. Furthermore, document US-A-2003/0186826 describes a dry cosmetic composition based on polymers and surfactants, to be administered to the skin or the hair with water. However, these documents never envisage the extemporaneous preparation of a cosmetic or dermatological product using kits comprising thin films and an aqueous composition, especially an aqueous composition in emulsion form.

Document FR-A-2 840 221 discloses films comprising active agents intended to be released into a liquid, this liquid possibly being water or a solvent such as oil or ethanol. However, the document never envisages the introduction of films into a complex medium such as an emulsion, or the provision of kits for obtaining a suitable product at the time of use.

A cosmetic kit for obtaining products comprising incompatible compounds, and moreover allowing the user to obtain a product that is adapted to his or her type of skin or to the desired aim at the time of use, either as regards the active agents contained or as regards the texture of the product or any other specific feature of the product in order for it to be adapted to the user's need at the time of use, which may be reflected by a particular fragrance or dye or by specific optical effects or by a given sun protection factor or a particular pH or any other characteristic that can vary according to the user's desire at the time he or she uses the kit, has never been described in the prior art. The present invention provides such a kit.

The kit according to the invention, which combines the use of at least one water-soluble anhydrous film based on polymers and of an aqueous composition, especially in emulsion form, allows the user to extemporaneously modify, e.g., the composition, the rheology, the colour or the active agent concentration of the final product. In addition, in the case of the application of active agents that are relatively unstable in liquid or semi-solid medium, their incorporation into the anhydrous film(s) to be dissolved extemporaneously ensures their efficacy. Similarly, good colloidal stability of the final product may be obtained by means of extemporaneous dissolution of an excipient whose performance qualities could not have been maintained over time in liquid medium.

The final cosmetic product is obtained by extemporaneous mixing of one or more water-soluble anhydrous films and of a suitable amount of the aqueous composition. The term “suitable amount” of aqueous composition means an amount such that the film(s) dissolve therein rapidly. This amount may range, for example, from 10 to 1000 mg, preferably from 50 to 800 mg and better still from 100 to 500 mg. The appropriate dose of aqueous composition may be obtained by using single-dose presentation forms, such as sachets, tubes, ampules, prefilled syringes, soft capsules, shells or trays made of hot-formed plastic. An appropriate dose may also be obtained from a multi-dose presentation by using a system that distributes a predefined dose. Such a system may be a pump-dispenser bottle, an aerosol, a pipette or a graduated syringe, or a dropper.

Other subjects will become apparent in the further detailed description that follows.

### A. Water-soluble Anhydrous Films

In the present patent application, the term “film” means a thin, palpable solid. The term “thin” means a solid with a maximum thickness of 1000 μm. This film generally has a size that is adequate to be able to be handled easily by the user. It may have a square, rectangular or disc shape, or any other shape. Each film preferably generally has a thickness of from 10 μm to 1000 μm, preferably from 20 to 500 μm and better still from 50 to 300 μm. It may have a surface area of from 0.25 to 25 cm² and preferably from 2 to 10 cm².

Moreover, in the present patent application, the term “anhydrous film” means a film comprising less than 10% by weight of water and preferably less than 5% by weight of water relative to the total weight of the film, and more preferably comprising no water.

In addition, in the present patent application, the term “water-soluble film” means a film which dissolves in water. This is a film comprising, or consisting, of one or more water-soluble or water-dispersible polymers. The term “water-soluble or water-dispersible” means polymers with a solubility in water, measured at 25° C., of at least 0.1 gram/litre (g/L) (production of a macroscopically isotropic and transparent, coloured or colourless solution). This solubility is preferably greater than or equal to 1 g/L. The polymers for making these films may be of synthetic or natural origin and, where appropriate, may be modified by means of chemical reactions. They may or may not be film-forming. These polymers should be physiologically acceptable, i.e. they should be compatible with the skin, mucous membranes, the hair and the scalp.

These water-soluble or water-dispersible polymers may be chosen, for example, from (1) polymers of protein type, such as wheat or soybean proteins; keratin, for example keratin hydrolysates and sulfonic keratins; casein; albumin; collagen; glutelin; glucagon; gluten; zein; gelatins and derivatives thereof; (2) polymers derived from chitin or from chitosan, such as anionic, cationic, amphoteric or nonionic chitin or chitosan polymers; (3) polysaccharide polymers such as, especially, (i) cellulose-based polymers, for instance hydroxyethylcellulose, hydroxypropylcellulose, hydroxypropylmethylcellulose, methylcellulose, ethylhydroxyethylcellulose, carboxymethylcellulose and quaternized cellulose derivatives; and (ii) starches and derivatives thereof; (4) acrylic polymers or copolymers such as polyacrylates, polymethacrylates and copolymers thereof; (5) vinyl polymers such as polyvinylpyrrolidones, copolymers of methyl vinyl ether and of maleic anhydride, the copolymer of vinyl acetate and of crotonic acid, copolymers of vinylpyrrolidone and of vinyl acetate, copolymers of vinylpyrrolidone and of caprolactam, polyvinyl alcohols; (6) polymers of natural origin, which are optionally modified, such as gum arabic, guar gum, xanthan derivatives or karaya gum; alginates, carrageenans, ulvanes and other algal colloids; glycoaminoglycans, hyaluronic acid and its derivatives; shellac, sandarac gum, dammar resins, elemi gums and copal resins; deoxyribonucleic acid; mucopolysaccharides such as hyaluronic acid, chondroitin sulphate; and mixtures of these polymers.

Water-soluble polymers that may also be mentioned include caprolactams, pullulan, pectin, mannan and galactomannans, and glucomannans, and derivatives thereof.

Needless to say, the films in accordance with the invention may comprise one or more of these polymers.

The amount of water-soluble polymer(s) in the anhydrous film generally ranges from 5% to 100% by weight, in particular from 10% to 95% by weight and more particularly from 20% to 90% by weight relative to the total weight of the film.

The films may be packaged in an article that facilitates their handling, such as the article described in patent application FR 0 351 002, the content of which is included in the present patent application by reference. The films may especially be packaged in a plastic dispensing box, in an individual sachet or in a blister pack, contained in the kit of the invention. The films may be packaged in a case of the type with a drawer or with a lid articulated on a base, the case possibly comprising means for facilitating the distribution of the articles. The distribution means may be of the type described, for example, in documents U.S. Pat. No. 2,973,882, GB-A-2 358 627, CH-A-461 025 or U.S. Pat. No. 6,578,732.

Besides the water-soluble polymer(s), the film may comprise one or more plasticizers chosen, for example, from glycerol, sorbitol, mono- and/or disaccharides, dipropylene glycol, butylene glycol, pentylene glycol and polyethylene glycols such as PEG-400. The amount of plasticizer(s) may range, for example, from 1% to 40% by weight and better still from 2% to 15% by weight relative to the total weight of the film.

In accordance with the invention, the film comprises at least one compound, especially a compound capable of modifying the aqueous composition. The expression “compound capable of modifying the aqueous composition” means any compound which, on mixing the film with the aqueous composition, will modify the composition, for example modify the rheology, the texture, the activity, the colour, the fragrance or the pH of the composition. This compound may simply be the water-soluble polymer present in the film, which, by virtue of its nature, will modify the rheology of the aqueous composition and give it a new texture, or it may be one or more compounds chosen from active agents or formulation adjuvants, for instance rheology agents, fragrances, dyestuffs, surfactants, antioxidants or pH regulators. Examples of these compounds will be detailed hereinbelow.

### B. Aqueous Composition

In the present patent application, the term “aqueous composition” means a composition comprising at least water.

Besides water, the aqueous composition may comprise a water-soluble organic solvent chosen, for example, from lower monoalcohols comprising from 1 to 8 carbon atoms and in particular 1 to 6 carbon atoms, for instance ethanol, isopropanol, propanol or butanol; polyethylene glycols comprising from 6 to 80 ethylene oxides; polyols, for instance propylene glycol, isoprene glycol, butylene glycol, glycerol or sorbitol; acetone; and mixtures thereof.

The composition is a more or less fluid composition, as opposed to a solid composition. In the present patent application, the term “more or less fluid composition” means a composition whose viscosity can be measured, and which ranges from liquid to semi-solid (cream or soft paste). The viscosity may range, for example, from 1 to 20 000 mPa·s (1 to 20 000 cpoises) and preferably from 1 to 15 000 mPa·s, this viscosity being measured at 25° C. using a Rheomat RM180 viscometer from Rheometric Scientific, this machine being equipped with a different rotor depending on the viscosities.

This composition comprises a physiologically acceptable support, i.e. a support that is compatible with the skin, mucous membranes, the hair and the scalp.

According to one preferred embodiment of the invention, the aqueous composition contains, besides water, at least one fatty substance. The term “fatty substance” means any water-insoluble compound, this compound possibly being an oil (liquid fatty substance) or a solid fatty substance. It is preferably an oil.

The aqueous composition may especially be in the form of an emulsion, compositions comprising an aqueous phase and an oily phase dispersed in each other, for example water-in-oil (W/O) or oil-in-water (O/W) or multiple (W/O/W or O/W/o) emulsions, chosen especially from standard emulsions or particular emulsions, for instance from:
O/W emulsions based on oily globules provided with a lamellar liquid crystal coating, as described in documents EP-A-641 557 and EP-A-705 593;
surfactant-free O/W emulsions stabilized with water-dispersible anionic polymers, such as those described in document EP-A-864 320;
O/W emulsions based on polymers derived from 2-acrylamido-2-methylpropanesulfonic acid (AMPS polymer), as described in document EP-A-815 844;
O/W emulsions stabilized with hydrophobic AMPS polymers, as described in documents EP-A-1 069 142, WO-A-2002/43689, WO-A-2002/44231, WO-A-2002/44271, WO-A-2002/44270, WO-A-2002/43686, WO-A-2002/44267, WO-A-2002/43688, WO-A-2002/43677, WO-A-2002/43687, WO-A-2002/44230;
fluid emulsions based on thermo-associative polymers, as described in documents EP-A-1 355 990, EP-A-1 355 625, EP-A-1 307 501 and EP-A-1 363 964;
O/W emulsions obtained via the PIT method (emulsion obtained by phase inversion. PIT=Phase Inversion Temperature), as described in documents WO-A-89/11907, DE-A-4 318 171 and EP-A-815 846;
nanoemulsions such as those described in patent applications EP-A-728 460, EP-A-780 114, EP-A-780 115, EP-A-879 589, EP-A-1 010 413, EP-A-1 010 414, EP-A-1 010 415, EP-A-1 010 416, EP-A-1 013 338, EP-A-1 016 453, EP-A-1 018 363, EP-A-1 020 219, EP-A-1 025 898, EP-A-1 120 102, EP-A-1 120 101, EP-A-1 160 005, EP-A-1 172 077 and EP-A-1 353 629.

The aqueous composition of the invention may especially be in the form of lotions (fluid emulsions).

The aqueous composition may also be in the form of foaming aqueous products, as described, for example, in documents EP-A-1 166 747, EP-A-1 172 096, EP-A-1 172 095, EP-A-1 174 122, EP-A-1 277 463, EP-A-1 295 594 and FR-A-2 824 262.

According to one particular embodiment of the invention, the aqueous composition used according to the invention is an emulsion. The proportion of the oily phase of the emulsion may range from 1% to 80% by weight and preferably from 1% to 50% by weight relative to the total weight of the composition. The oils, emulsifiers and co-emulsifiers optionally present, used in the composition in emulsion form, are chosen from those conventionally used in cosmetics or dermatology. When they are present, the emulsifier and the co-emulsifier are generally in a proportion ranging from 0.2% to 30% by weight, preferably from 0.3% to 20% by weight and better still from 0.5% to 15% by weight relative to the total weight of the composition. The emulsion may also contain lipid vesicles.

The emulsions generally contain at least one emulsifier chosen from amphoteric, anionic, cationic and nonionic emulsifiers, used alone or as a mixture. The emulsifiers are chosen in an appropriate manner according to the continuous phase of the emulsion to be obtained (W/O or O/W). When the emulsion is a multiple emulsion, it generally comprises an emulsifier in the primary emulsion and an emulsifier in the external phase into which the primary emulsion is introduced.

As emulsions that may be used for the preparation of the W/O emulsions, examples that may be mentioned include alkyl esters or ethers of sorbitan, of glycerol or of sugars; silicone surfactants, for instance dimethicone copolyols, such as the mixture of cyclomethicone and of dimethicone copolyol, sold under the names DC 5225 C and DC 3225 C by the company Dow Corning, and alkyldimethicone copolyols such as the laurylmethicone copolyol sold under the name “Dow Corning 5200 Formulation Aid” by the company Dow Corning, cetyldimethicone copolyol sold under the name Abil EM 90® by the company Goldschmidt and the mixture of polyglyceryl-4 isostearate/cetyldimethicone copolyol/hexyl laurate sold under the name Abil WE 09® by the company Goldschmidt. One or more co-emulsifiers may also be added thereto, which may be chosen advantageously from the group comprising branched-chain fatty acid esters of polyols, and especially branched-chain fatty acid esters of glycerol and/or of sorbitan, for example polyglyceryl isostearate, such as the product sold under the name Isolan GI 34 by the company Goldschmidt, sorbitan isostearate, such as the product sold under the name Arlacel 987 by the company ICI, and sorbitan glyceryl isostearate, such as the product sold under the name Arlacel 986 by the company ICI, and mixtures thereof.

As other emulsifying agents that may be used for the preparation of O/W emulsions, examples that may be mentioned include nonionic emulsifiers such as oxyalkylenated (more particularly polyoxyethylenated) fatty acid esters of polyols, for example polyethylene glycol stearates, for instance PEG-100 stearate, PEG-50 stearate and PEG-40 stearate; and mixtures thereof, such as the mixture of glyceryl monostearate and of polyethylene glycol stearate (100 EO) sold under the name Simulsol 165 by the company SEPPIC; oxyalkylenated fatty acid esters of sorbitan comprising, for example, from 20 to 100 EO, for example those sold under the trade names Tween 20 or Tween 60 by the company Uniqema; oxyalkylenated (oxyethylenated and/or oxypropylenated) fatty alcohol ethers; alkoxylated or non-alkoxylated sugar esters, for instance sucrose stearate and PEG-20 methylglucose sesquistearate; sorbitan esters such as the sorbitan palmitate sold under the name Span 40 by the company Uniqema; esters of diacid and of fatty alcohol, such as dimyristyl tartrate; mixtures of these emulsifiers, for instance the mixture of glyceryl stearate and of PEG-100 stearate, sold under the name Arlacel 165 by the company Uniqema; and mixtures comprising these emulsifiers, such as the mixture of dimyristyl tartrate, cetearyl alcohol, Pareth-7 and PEG-25 laureth-25, sold under the name Cosmacol PSE by the company Sasol (CTFA name: dimyristyl tartrate/cetearyl alcohol/12-15 Pareth 7/PPG 25 laureth 25).

Co-emulsifiers may be added to these emulsifiers, for instance fatty alcohols comprising from 8 to 26 carbon atoms, for instance cetyl alcohol, stearyl alcohol and the mixture thereof (cetearyl alcohol), octyldodecanol, 2-butyloctanol, 2-hexyldecanol, 2-undecylpentadecanol or oleyl alcohol, or fatty acids.

Emulsions free of emulsifying surfactants or comprising less than 0.5% of emulsifying surfactants relative to the total weight of the composition may also be prepared, by using suitable compounds, for example polymers having emulsifying properties, such as the polymers sold under the names Carbopol 1342 and Pemulen by the company Noveon; or polymers in emulsion form such as the product sold under the name Sepigel 305 by the company Seppic (INCI name: polyacrylamide/C13-C14 isoparaffin/laureth-7); particles of ionic or nonionic polymers, more particularly particles of anionic polymer such as, especially, isophthalic acid or sulfoisophthalic acid polymers, and in particular the phthalate/sulfoisophthalate/glycol copolymers (for example diethylene glycol/phthalate/isophthalate/1,4-cyclohexanedimethanol (INCI name: diglycol/CHDM/isophthalates/SIP copolymer) sold under the names Eastman AQ polymer (AQ35S, AQ38S, AQ55S and AQ48 Ultra) by the company Eastman Chemical. Emulsifier-free emulsions stabilized with silicone particles or metal oxide particles such as TiO₂ or the like may also be prepared.

When the aqueous composition is in emulsion form, it comprises at least one oily phase that contains at least one oil, especially a cosmetic oil. The term “oil” means a fatty substance that is liquid at room temperature (25° C.).

As oils that may be used in the composition of the invention, examples that may be mentioned include:
hydrocarbon-based oils of animal origin, such as perhydrosqualene (or squalane);
hydrocarbon-based oils of plant origin, such as liquid triglycerides of fatty acids comprising from 4 to 10 carbon atoms, for instance heptanoic or octanoic acid triglycerides, or alternatively oils of plant origin, for example sunflower oil, corn oil, soybean oil, marrow oil, grapeseed oil, sesame seed oil, hazelnut oil, apricot oil, macadamia oil, arara oil, coriander oil, castor oil, avocado oil, jojoba oil, shea butter oil, or caprylic/capric acid triglycerides, for instance those sold by the company Stearineries Dubois or those sold under the names Miglyol 810, 812 and 818 by the company Dynamit Nobel;
synthetic esters and ethers, especially of fatty acids, for instance the oils of formulae R¹COOR² and R¹OR² in which R¹ represents a fatty acid residue comprising from 8 to 29 carbon atoms and R² represents a branched or unbranched hydrocarbon-based chain comprising from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate and fatty alcohol heptanoates, octanoates and decanoates; polyol esters, for instance propylene glycol dioctanoate, neopentyl glycol diheptanoate and diethylene glycol diisononanoate; pentaerythritol esters, for instance pentaerythrityl tetraisostearate; lipophilic derivatives of amino acids, such as isopropyl lauroyl sarcosinate (INCI name: isopropyl lauroyl sarcosinate) sold under the name Eldew SL 205 by the company Ajinomoto;
linear or branched hydrocarbons of mineral or synthetic origin, such as mineral oils (mixtures of petroleum-derived hydrocarbon-based oils; INCI name: mineral oil), volatile or non-volatile liquid paraffins, and derivatives thereof, petroleum jelly, polydecenes, isohexadecane, isododecane, hydrogenated isoparaffin such as the Parleam® oil sold by the company NOF Corporation (INCI name: hydrogenated polyisobutene);
silicone oils, for instance volatile or non-volatile polymethylsiloxanes (PDMS) comprising a linear or cyclic silicone chain, which are liquid or pasty at room temperature, especially cyclopolydimethylsiloxanes (cyclomethicones) such as cyclopentasiloxane and cyclohexadimethylsiloxane; polydimethylsiloxanes comprising alkyl, alkoxy or phenyl groups, which are pendent or at the end of a silicone chain, these groups comprising from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes 2-phenylethyltrimethyl siloxysilicates and polymethylphenylsiloxanes;
fluoro oils such as partially hydrocarbon-based and/or partially silicone-based fluoro oils, for instance those described in document JP-A-2 295 912;
ethers such as dicaprylyl ether (CTFA name: dicaprylyl ether); and C₁₂-C₁₅ fatty alcohol benzoates (Finsolv TN from Finetex);
mixtures thereof.

The aqueous composition may also contain any suitable additive compound. It may contain, for example, one or more compounds chosen from formulation adjuvants and/or active agents different from those present in the film(s).

One of the advantages of the kit according to the invention is, for example, the ability to obtain a cosmetic product comprising incompatible active agents, one or more compatible active agents being present in the aqueous composition and one or more different active agents that are incompatible with those of the aqueous composition being present in the anhydrous film(s). The extemporaneous mixing of the aqueous composition and of the film(s) then results in a product comprising incompatible active agents, their incompatibility not presenting any drawback since the product is used immediately.

### C. Compounds

The water-soluble anhydrous film comprises at least one compound, especially a compound capable of modifying the aqueous composition. This compound may simply be the polymer constituting the film, this polymer being capable of modifying the rheology of the composition, or alternatively the film may contain one or more other compounds. These compounds may be chosen especially from cosmetic active agents, formulation adjuvants and mixtures thereof. It is also possible to use several films, one comprising a formulation adjuvant, the other a different formulation adjuvant or an active agent, and yet another possibly comprising another active agent and/or another formulation adjuvant.

The term “cosmetic active agents” means any compound that will have a beneficial effect on the keratin material onto which the final product is applied.

The term “formulation adjuvant” means any compound capable of modifying the appearance of the aqueous composition, for example the rheology, the texture, the colour, the fragrance or the pH of the aqueous composition.

Moreover, as indicated above, the aqueous composition may also contain one or more compounds chosen from cosmetic active agents and formulation adjuvants, and mixtures thereof.

Thus, these compounds may be incorporated either into the aqueous composition or into one or more films, which makes it possible to solve the possible problems of physicochemical incompatibility of these compounds, and also to have final products that are suited to the need of the user. This also makes it possible to obtain products whose concentrations of active agents are higher than the concentrations that might be obtained by using only one composition.

As indicated above, the kit according to the invention has the advantage of allowing the preparation of cosmetic or dermatological products comprising incompatible compounds. It also has the advantage of allowing the production of cosmetic or dermatological products comprising compounds that are sensitive to an external stimulus, especially sensitive to water, to oxidation, to light and/or to an increase in temperature. These compounds sensitive to an external stimulus (water, oxidizing agents such as air, light and/or temperature) are unstable and undergo, upon exposure to such an external stimulus, degradation via a mechanism of chemical hydrolysis, oxidation, photolysis or photodegradation, or alternatively of ion exchange, which leads to cosmetic compositions that become inefficient and/or whose appearance, odour and/or feel are unacceptable to the consumer. Such is therefore the case, for example, for ascorbic acid, which, as a result of this degradation, has a tendency to yellow compositions comprising it. The fact of placing these sensitive compounds in an anhydrous film and of not incorporating them into the aqueous composition until the time of use makes it possible to avoid all these problems.

Besides ascorbic acid, compounds sensitive to an external stimulus that may be mentioned include retinol (vitamin A) and its derivatives; urea; DHA (dihydroxyacetone); rutin; enzymes such as lipase, protease, phospholipase and cellulases; natural extracts such as green tea, extract of balm, extract of thyme, procyannidol oligomers (PCO) such as hawthorn PCO, pine PCO and grape PCO; certain acids such as kojic acid, caffeic acid, retinoic acid and its derivatives, benzene-1,4-bis(3-methylidene-10-camphorsulfonic acid); carotenoids such as carotenes, for instance α-, β- and γ-carotenes, β,φ-carotene, ξ-carotene, β,γ-carotene and lycopene (ψ,ψ-carotene); polyunsaturated fatty acids such as gamma-linolenic acid.

The compounds that may be used in the kit according to the invention may be chosen especially from moisturizers, anti-seborrhoeic agents, anti-ageing active agents, antimicrobial active agents, anti-inflammatory or calmative active agents, lipolytic or slimming active agents, fillers, sunscreens, skin-colouring or hair-colouring agents, concealing active agents, antiperspirant active agents, deodorant active agents, hair-treating active agents, hair-removing agents, pigments, dyes, polymers, fragrances, electrolytes, pH adjusters and preserving agents, and mixtures thereof.

I) Moisturizers, for instance sodium lactate; polyols, and in particular glycerol, sorbitol and polyethylene glycols; mannitol; amino acids; hyaluronic acid; lanolin; urea and mixtures comprising urea, such as NMF (“Natural Moisturizing Factor”); petroleum jelly; N-lauroylpyrrolidonecarboxylic acid and its salts; essential fatty acids; essential oils; and mixtures thereof.
II) Anti-seborrhoeic agents, chosen, for example, from:
sulfur and sulfur derivatives;
zinc salts such as zinc lactate, gluconate, pidolate, carboxylate, salicylate and/or cysteate;
selenium chloride;
vitamin B6 or pyridoxine;
the mixture of capryloyl glycine, of sarcosine and of extract of Cinnamomum zeylanicum sold especially by the company SEPPIC under the trade name Sepicontrol A5®;
an extract of Laminaria saccharina sold especially by the company SECMA under the trade name Phlorogine®;
an extract of Spiraea ulmaria sold especially by the company Silab under the trade name Sebonormine®;
plant extracts of the species Arnica montana, Cinchona succirubra, Eugenia caryophyllata, Humulus lupulus, Hypericum perforatum, Mentha piperita, Rosmarinus officinalis, Salvia officinalis and Thymus vulgaris, which are all sold, for example, by the company Maruzen;
an extract of Serenoa repens sold especially by the company Euromed;
extracts of plants of the genus Silybum;
plant extracts comprising sapogenins and in particular extracts of diosgenin-rich or hecogenin-rich Dioscorea plants;
extracts of Eugenia caryophyllata comprising eugenol and eugenyl glucoside;
and mixtures thereof.
   III) Anti-ageing active agents which may be chosen from any active agent capable of treating or preventing any sign of ageing of the skin. They may be chosen, for example, from free-radical scavengers, keratolytic agents, vitamins, anti-elastase and anti-collagenase agents, proteins, fatty acid derivatives, steroids, trace elements, bleaching agents, algal and plankton extracts, enzymes and coenzymes, flavonoids, ceramides, tensioning agents and muscle relaxants, and mixtures thereof.
   1) Free-radical scavengers and antioxidants that may especially be mentioned include phosphonic acid derivatives such as ethylenediaminetetra(methylenephosphonic acid), hexamethylenediaminetetra(methylene phosphonic acid), diethylenetriaminepenta(methylenephosphonic acid and salts thereof, in particular the sodium salts thereof; ethylenediaminetetraacetic acid and its salts, such as the sodium salt; guanosine; superoxide dismutase; tocopherol (vitamin E) and its derivatives (acetate); ethoxyquine; lactoferrin; lactoperoxidase, and nitroxide derivatives; superoxide dismutases; glutathione peroxidase; plant extracts with free-radical-scavenging activity, such as the aqueous extract of wheatgerm sold by the company Silab under the reference Detoxiline; green tea; and mixtures thereof.
   2) Examples of keratolytic agents that may be mentioned include α-hydroxy acids, especially acids derived from fruit, for instance glycolic acid, lactic acid, malic acid, citric acid, tartaric acid and mandelic acid, derivatives thereof and mixtures thereof; β-hydroxy acids, for instance salicylic acid and its derivatives such as 5-n-octanoylsalicylic acid or 5-n-dodecanoylsalicylic acid; α-keto acids, for instance ascorbic acid or vitamin C and derivatives thereof, such as its salts, for instance sodium ascorbate and magnesium or sodium ascorbyl phosphate; esters thereof, for instance ascorbyl acetate, ascorbyl palmitate and ascorbyl propionate, or sugars thereof, for instance glycosyl ascorbic acid and mixtures thereof; β-keto acids; retinoids, for instance retinol (vitamin A) and its esters, retinal, retinoic acid and its derivatives, and also the retinoids described in documents FR-A-2 570 377, EP-A-199 636, EP-A-325 540 and EP-A-402 072; adapalene; carotenoids; and mixtures thereof.
   3) Vitamins, besides vitamins A, E and C indicated above, that may be mentioned in particular include vitamin B3 (or vitamin PP or niacinamide) and its derivatives (tocopheryl nicotinate, nicotinyl alcohol esters and esters of carboxylic acids, 2-chloronicotinamide, 6-methylnicotinamide, 6-aminonicotinamide, N-methylnicotinamide, N,N-dimethylnicotinamide, N-(hydroxymethyl)nicotinamide, quinolinic acid imide, nicotinanilide, N-benzylnicotinamide, N-ethylnicotinamide, nifenazone, nicotinaldehyde, isonicotinic acid, methylisonicotinic acid, thionicotinamide, nialamide, 2-mercaptonicotinic acid, nicomol and niaprazine); vitamin B5 (or panthenol or panthenyl alcohol or 2,4-dihydroxy-N-(3-hydroxypropyl)-3,3-dimethylbutanamide) in its various forms: D-panthenol, DL-panthenol), and derivatives and analogues thereof, such as calcium panthotenate, panthetine, pantotheine, panthenyl ethyl ether, pangamic acid, pyridoxine, pantoyl lactose, and natural compounds comprising them, such as royal jelly; vitamin D and its analogues such as those described in document WO-A-00/26167; vitamin F or its analogues such as mixtures of unsaturated acids comprising at least one double bond, and especially mixtures of linoleic acid, of linolenic acid and of arachidonic acid, or compounds comprising them and especially oils of plant origin comprising them, for instance jojoba oil, and mixtures thereof.
   4) Anti-elastase agents that may especially be mentioned include peptide derivatives and especially peptides from legume seeds such as those sold by Laboratoires Seriobiologiques de Nancy under the reference Parelastyl; the N-acylamino amide derivatives described in patent application FR-A-2 180 033, for instance ethyl {2-[acetyl(3-trifluoromethylphenyl)-amino]-3-methylbutyrylamino}acetate and {2-[acetyl(3-trifluoromethylphenyl)amino]-3-methylbutyrylamino}acetic acid, and mixtures thereof.
   5) Anti-collagenase agents that may be mentioned include metalloprotease inhibitors, such as ethylenediamine acid (EDTA) and cysteine, and mixtures thereof.
   6) Examples of proteins that may be mentioned include wheat or soybean proteins, hydrolysates thereof, for instance the products sold by the company Silab under the reference Tensine, and mixtures thereof.
   7) Fatty acid derivatives that may especially be mentioned include polyunsaturated phospholipids including the phospholipids of essential fatty acids of octopus, and mixtures thereof.
   8) Examples of steroids that may be mentioned include DHEA or dehydroepiandrosterone, biological precursors thereof, metabolites thereof, and mixtures thereof. The term “biological precursors” of DHEA especially means Δ5-pregnenolone, 17α-hydroxypregnenolone and 17α-hydroxypregnenolone sulfate. The term “DHEA derivatives” means both the metabolic derivatives thereof and the chemical derivatives thereof. Metabolic derivatives that may especially be mentioned include Δ5-androstene-3,17-diol and especially 5-androstene-3β,17β-diol, Δ4-androstene-3,17-dione, 7-hydroxy DHEA (7α-hydroxy DHEA or 7β-hydroxy DHEA), 7-keto DHEA, which is itself a metabolite of 7β-hydroxy DHEA, and benzoyl DHEA.
   9) Examples of trace elements that may be mentioned include copper, zinc, selenium, iron, magnesium and manganese, and mixtures thereof.
   10) Examples of bleaching or depigmenting agents that may be mentioned include kojic acid and its derivatives; hydroquinone and its derivatives such as arbutin and its esters; vitamin C and its derivatives such as magnesium ascorbyl phosphate; salts such as calcium D-pantetheine sulfonate; ellagic acid and its derivatives; rucinol; linoleic acid and its derivatives; plant extracts and especially extracts of liquorice, of mulberry or of skullcap, glutathione and precursors thereof; cysteine and precursors thereof; the aminophenol-based compounds described in document WO-A-99/10318, such as, especially, N-ethyloxycarbonyl-4-aminophenol, N-ethyloxycarbonyl-O-ethyloxycarbonyl-4-aminophenol, N-cholesteryloxycarbonyl-4-aminophenol and N-ethylaminocarbonyl-4-aminophenol; and mixtures of these compounds.
   11) Algal extracts that may be mentioned include extracts of red or brown algae, for example the extract of brown algae of the Laminaria family, for instance extracts of the species Laminaria digitata, and more particularly the product sold by the company Codif under the name Phycosaccharides, which is a concentrated solution of an oligosaccharide comprising a sequence of two uric acids: mannuronic acid and guluronic acid.
   12) A plankton extract that may be mentioned is the plankton as an aqueous dispersion (CTFA name: Vitreoscilla ferment) sold under the name Mexoryl SAH by the company Chimex.
   13) Enzymes that may be used include any enzyme of animal, microbiological (bacterial, fungal or viral) or synthetic origin (obtained via chemical or biotechnological synthesis), in pure crystalline form or in a form diluted in an inert diluent. Examples that may be mentioned include lipases, proteases, phospholipases, laccases, cellulases, peroxidases, especially lactoperoxidases, catalases, superoxide dismutases, or plant extracts comprising the abovementioned enzymes, and mixtures thereof. They may be chosen, for example, from the product sold under the trade name “Subtilisin SP 554” by the company Novo Nordisk and the product sold under the trade name “Lysoveg LS” by the company Laboratoires Seriobiologiques de Nancy.
   14) Coenzymes that may especially be used include ubiquinone or coenzyme Q10, which belongs to the family of benzoquinones comprising an alkylene chain, and coenzyme R, which is biotin (or vitamin H), and mixtures thereof.
   15) Examples of flavonoids that may be mentioned include isoflavonoids, which constitute a subclass of flavonoids, formed from a 3-phenylchroman skeleton, which may comprise various substituents and different levels of oxidation. The term “isoflavonoid” includes several classes of compounds, among which mention may be made of isoflavones, isoflavanones, rotenoids, pterocarpans, isoflavanes, isoflavan-3-enes, 3-arylcoumarins, 3-aryl-4-hydroxycoumarins, coumestanes, coumaronochromones, α-methyldeoxybenzoins, 2-arylbenzofurans, and mixtures thereof. The isoflavanoids may be of natural or synthetic origin. The term “natural origin” means the isoflavonoid in pure form or as a solution in different concentrations, obtained via various extraction processes from a component, generally a plant, of natural origin. The term “synthetic origin” means the isoflavonoid in pure form or as a solution in different concentrations, obtained via chemical synthesis. Isoflavonoids of natural origin that may be mentioned include daidzin, genistin, daidzein, formononetin, cuneatin, genistein, isoprunetin and prunetin, cajanine, orobol, pratensein, santal, junipegenin A, glycitein, afrormosin, retusin, tectorigenin, irisolidone, jamaicin, and also analogues and metabolites thereof.
   16) Ceramides that may be used include any type of ceramide of natural or synthetic origin, for example of type II, of type III, of type IV, of type V or of type VI, and mixtures thereof. Examples of ceramides that may be mentioned include N-oleoyldihydrosphingosine, N-stearoylphytosphingosine, N-α-hydroxybehenoyldihydrosphingosine, N-α-hydroxypalmitoyldihydrosphingosine, N-linoleoyldihydrosphingosine, N-palmitoyldihydrosphingosine, N-stearoyldihydrosphingosine and N-behenoyldihydrosphingosine, and mixtures thereof.
   17) Examples of tensioning agents that may be mentioned include:
synthetic polymers;
polymers of natural origin;
mixed silicates;
wax microparticles;
colloidal particles of mineral fillers.

The synthetic polymers that may be used as tensioning agents may be chosen from:
polyurethane polymers and copolymers;
acrylic polymers and copolymers;
sulfoisophthalic acid polymers;
grafted silicone polymers;
water-soluble or water-dispersible polymers comprising water-soluble or water-dispersible units and units with an LCST (Lower Critical Solution Temperature).

The polyurethane copolymers, the acrylic copolymers and the other synthetic polymers that may be used as tensioning agents may be chosen especially from polycondensates, hybrid polymers and interpenetrating polymer networks (IPNs). The term “interpenetrating polymer network” means a mixture of two intermeshed polymers, obtained by simultaneous polymerization and/or crosslinking of two types of monomer, the mixture obtained having a single glass transition temperature. Examples of IPNs that are suitable as tensioning polymers, and also the process for preparing them, are described, for example, in documents U.S. Pat. No. 6,139,322 and U.S. Pat. No. 6,465,001. Preferably, the IPN comprises at least one polyacrylic polymer, and it more preferably also comprises at least one polyurethane or a copolymer of vinylidene fluoride and of hexafluoropropylene. According to one preferred embodiment, the IPN comprises a polyurethane polymer and a polyacrylic polymer. Such IPNs are especially those of the Hybridur series sold by the company Air Products. An IPN that is particularly preferred as tensioning polymer is in the form of an aqueous dispersion of particles with a weight-average size of between 90 and 110 nm and a number-average size of about 80 nm. This IPN preferably has a glass transition temperature, Tg, ranging from about −60° C. to +100° C. An IPN of this type is sold especially by the company Air Products under the trade name Hybridur X-01602. Another IPN that is suitable for use in the present invention has the reference Hybridur X18693-21 or Hybridur 875 polymer dispersion.

Other IPNs that are suitable as tensioning polymers include the IPNs consisting of a mixture of a polyurethane with a copolymer of vinylidene fluoride and of hexafluoropropylene, especially those prepared as described in document U.S. Pat. No. 5,349,003. As a variant, they are commercially available in the form of a colloidal dispersion in water, in a ratio of the fluoro copolymer to the acrylic polymer of between 70:30 and 75:25, under the trade names Kynar RC-10,147 and Kynar RC-10,151 from the company Atofina.

Examples of grafted silicone polymers are indicated in document EP-A-1 038 519, which is incorporated herein by reference. A preferred example of a grafted silicone polymer is polysilicone-8 (CTFA name), which is a polydimethylsiloxane on which are grafted, via a linking chain of thiopropylene type, mixed polymer units of the poly(meth)acrylic acid type and of the polyalkyl (meth)acrylate type. A polymer of this type is especially available under the trade name VS 80 (at 10% in water) or LO 21 (in pulverulent form) from the company 3M. It is a copolymer of polydimethylsiloxane comprising propylthio, methyl acrylate, methyl methacrylate and methacrylic acid groups.

The abovementioned synthetic polymers may be in the form of latices. As suitable latices that may be used as tensioning agents, mention may be made especially of polyester-polyurethane and polyether-polyurethane dispersions, such as those sold under the names Avalure UR410 and UR460 by the company Noveon, and under the names Neorez R974, Neorez R981 and Neorez R970, and also acrylic copolymer dispersions such as those sold under the name Neocryl XK-90 by the company Avecia.

Finally, synthetic polymers that are suitable as tensioning polymers may be water-soluble or water-dispersible polymers comprising water-soluble or water-dispersible units and comprising units with an LCST, the units with an LCST having, in particular, a demixing temperature in water of from 5 to 40° C. at a mass concentration of 1%. This type of polymer is more fully described in document FR-A-2 819 429.

The polymers of natural origin that may be used as tensioning agents may be chosen from:
plant proteins and plant protein hydrolysates;
polysaccharides of plant origin, optionally in the form of microgels, such as starch;
latices of plant origin.

As examples of plant proteins and plant protein hydrolysates that may be used as tensioning agents, mention may be made of proteins and protein hydrolysates of corn, rye, Triticum aestivum, buckwheat, sesame, spelt, pea, bean, lentil, soybean and lupin.

The polysaccharides that may be used as tensioning agents may be chosen from polysaccharides of natural origin capable of forming heat-reversible or crosslinked gels and solutions. The term “heat-reversible” means that the gel state of these polymer solutions is obtained reversibly, once the solution has cooled below the characteristic gel point of the polysaccharide used. As polysaccharides of natural origin of this type, mention may be made of carrageenans and most particularly kappa-carrageenan and iota-carrageenan; agars, gellans; alginates; pectins; chitosans and derivatives thereof; pullulans and derivatives thereof. These tensioning polysaccharides may be present in the form of microgels as described in document FR-A-2 829 025.

The polysaccharides may also be chosen from starch and its derivatives. The starch may be of any origin: for example rice, corn, potato, cassaya, pea, Triticum aestivum or oat, and it may be natural or optionally modified by means of a treatment such as crosslinking, acetylation or oxidation. It may optionally be grafted. As a starch that may be used as a tensioning agent, an example that may be mentioned is the product sold by the company Lambert-Rivière under the name Remi Dri.

Another class of tensioning agents that may be used according to the invention consists of mixed silicates. This term means any silicate of natural or synthetic origin comprising several types of cations chosen from alkali metals (for example Na, Li or K) or alkaline-earth metals (for example Be, Mg or Ca) and transition metals. Phyllosilicates are preferably used, i.e. silicates having a structure in which the SiO₄ tetrahedra are organized in lamellae between which the metal cations are enclosed. One family of silicates that is particularly preferred as tensioning agents is the laponite family. Laponites are magnesium lithium sodium silicates that have a layer structure similar to that of montmorillonites. Laponite is the synthetic form of the natural mineral known as hectorite. The laponite sold under the name Laponite XLS or Laponite XLG by the company Rockwood may be used, for example.

Yet another class of tensioning agents consists of wax microparticles. These are particles with a diameter generally of less than 5 μm, or better still 0.5 μm, and consisting essentially of a wax or a mixture of waxes chosen, for example, from carnauba wax, candelilla wax or esparto grass wax. The melting point of the wax or of the mixture of waxes is preferably between 50° C. and 150° C.

As another variant, tensioning agents that may be used are colloidal particles of mineral fillers. The term “colloidal particles” means colloidal particles dispersed in an aqueous, aqueous-alcoholic or alcoholic medium, having a number-average diameter of between 0.1 and 100 nm and preferably between 3 and 30 nm. Examples of mineral fillers that may be mentioned include silica, cerium oxide, zirconium oxide, alumina, calcium carbonate, barium sulfate, calcium sulfate, zinc oxide and titanium dioxide. A mineral filler that is particularly preferred is silica. Colloidal silica particles are especially available in the form of an aqueous dispersion of colloidal silica from the company Catalysts & Chemicals under the trade names Cosmo S-40 and Cosmo S-50. Silica-alumina composite colloidal particles may also be used, such as those sold by the company Grace under the names Ludox AM, Ludox HSA and Ludox TMA.

18) As muscle relaxants, which are agents for smoothing out expression wrinkles, examples that may be mentioned include sapogenins (see patent application EP-A-1 352 643); adenosine (see patent application FR-02/14828); antagonists of receptors associated with the calcium channels (see patent application FR-A-2 793 681) and in particular manganese and its salts (see patent application FR-A-2 809 005) and alverine (see patent application FR-A-2 798 590); agonists of receptors associated with the chlorine channels, including glycine (see patent application EP-A-0 704 210) and certain extracts of Iris pallida (see patent application FR-A-2 746 641).
IV) Antimicrobial or antifungal active agents, especially 2,4,4′-trichloro-2′-hydroxydiphenyl ether (or triclosan), 3,4,4′-trichlorobanilide (or triclocarban), phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanalide, octopirox, octoxyglycerol, octanoylglycine, caprylyl glycol, 10-hydroxy-2-decanoic acid, dichlorophenylimidazole dioxolane and derivatives thereof described in document WO-A-93/18743, farnesol, phytosphingosines, selenium derivatives, zinc pyrithione, tetracyclines, for instance erythromycin, and mixtures of these compounds.
V) Anti-inflammatory or calmative active agents such as pentacyclic triterpenes and plant extracts (e.g.: Glycyrrhiza glabra) comprising them, for instance β-glycyrrhetinic acid and salts and/or derivatives thereof (glycyrrhetinic acid monoglucuronide, stearyl glycyrrhetinate or 3-stearoyloxyglycyrrhetic acid); ursolic acid and its salts; oleanolic acid and its salts; betulinic acid and its salts; extracts of the pea Paeonia suffruticosa, of lactiflora, of Laminaria saccharina, of camomile, of Pygeum, of Boswellia serrata, of Centipeda cunnighami, of Helianthus annuus, of Linum usitatissimum, of Cola nitida, of clove, of Epilobium angustifolium or of Bacopa monieri; salicylic acid salts, and in particular zinc salicylate; phycosaccharides from the company Codif; Canola oil; bisabolol; allantoin; Sepivital EPC (phosphoric diester of vitamin E and C) from the company SEPPIC; omega-3 unsaturated oils such as musk rose oil, blackcurrant oil, Echium oil, or fish oil; capryloyl glycine; Seppicalm VG (sodium palmitoylproline and Nymphea alba) from the company SEPPIC; tocotrienols; piperonal, Aloe vera; phytosterols.
VI) Lipolytic or slimming active agents, i.e. active agents with direct or indirect favourable activity on reducing adipose tissue, such as:
xanthine derivatives, for instance caffeine and its derivatives, especially the 1-hydroxyalkylxanthines described in document FR-A-2 617 401, caffeine citrate, theophylline and its derivatives, theobromine, acefylline, aminophylline, chloroethyltheophylline, diprofylline, diniprophylline, etamiphylline and its derivatives, etofylline and proxyphylline; or combinations comprising xanthine derivatives, for instance the combination of caffeine and silanol (methylsilanetriol derivative of caffeine), for example the product sold by the company Exsymol under the name cafeisilane C; or compounds of natural origin comprising xanthine bases and especially caffeine, such as extracts of tea, of coffee, of guarana, of mate, of cola (Cola nitida) and especially the dry extract of guarana fruit (Paulina sorbilis) comprising 8 to 10% caffeine; ephedrine and its derivatives, which may especially be in natural form in plants such as Ma Huang (Ephedra plant);
plant extracts and extracts of marine origin, which are either active on the receptors to be inhibited, such as β-2 blockers, NPY blockers (described in document EP-A-838 217), or inhibit the synthesis of LDL or VLDL receptors, or are active in stimulating the receptors and the G proteins, leading to the activation of adenylcyclase. Examples of plant extracts of this type that may be mentioned include:
Garcinia cambogia,
extracts of Bupleurum chinensis,
extracts of climbing ivy (Hedera helix), of arnica (Arnica Montana L), of rosemary (Rosmarinus officinalis N), of marigold (Calendula officinalis), of sage (Salvia officinalis L), of ginseng (Panex ginseng), of St. John's Wort (Byperycum perforatum), of ruscus (Ruscus aculeatus L), of ulmaria (Filipendula ulmaria L), of orthosiphon (Orthosiphon stamincus Benth), of birch (Betula alba), of pumpwood and of argan tree,
extracts of ginkgo biloba,
extracts of horsetail,
extracts of escine,
extracts of cangzhu,
extracts of Chrysanthellum indicum,
extracts of diosgenin-rich dioscoria plants or pure diosgenin or hecogenin and derivatives thereof,
extracts of plants of the genus Armeniacea, Atractylodis platicodon, Sinom-menum, Pharbitidis and Flemingia,
extracts of Coleus such as C. forskohlii, C. blumei, C esquirolii, C. scutellaroides, C. xanthantus and C. barbatus, such as the extract of Coleus barbatus root comprising 60% forskolin,
extracts of ballota,
extracts of Guioa, of Davallia, of Terminalia, of Barringtonia, of Trema or of Antirobia.

Extracts of marine origin that may be mentioned include:
extracts of algae or of phytoplankton, such as rhodysterol or the extract of Laminaria digitata sold under the name Phycox 75 by the company Secma, the alga skeletonema described in document FR-A-2 782 921 or the diatoms described in document FR-A-2 774 292.
   VII) Fillers, and especially those that provide optical effects, i.e. fillers capable of imparting, after application to the skin, the following optical effects:
matting or a reduction in gloss;
soft focus effect: the term “soft focus effect” means the visible reduction in wrinkles, pores and irregularities of the skin microrelief, this reduction being obtained instantaneously after applying the cosmetic composition to skin presenting imperfections associated with an irregular microrelief;
homogenization and lightening of the complexion: the term “homogeneity of the complexion” means the visible reduction in dyschromia and pigmentary irregularities of the skin, obtained instantaneously after applying the cosmetic composition to skin presenting these imperfections.

The fillers are solid, generally white particles, which are insoluble in the medium of the composition.

Among the fillers with an optical effect that may be used in the invention, mention may be made of mineral fillers (amorphous titanium dioxide or titanium dioxide crystallized in rutile and/or anatase form, zinc oxide, iron oxide, cerium oxide, silica, alumina, boron nitride, talc, sericite, mica, etc), which may be coated or uncoated, and also composite fillers, nacres, clays, starch and its derivatives, aqueous dispersions of acrylic styrene, melamine-formaldehyde or urea-formaldehyde resin particles, aqueous dispersions of polytetrafluoroethylene (PTFE), wax microdispersions, polyvinylpyrrolidone/1-tricontene copolymers, silicone waxes and resins, organopolysiloxane particles, microspheres of expanded terpolymer of vinylidene chloride, of acrylonitrile and of methacrylate, Nylon particles, cellulose microbeads, fibres, hollow hemispherical silicone particles such as those sold under the names NLK-500 and NLK-503 by the company Takemoto Oil and Fat.

VIII) Sunscreens which may be chosen from the UVA and UVB chemical screening agents or the physical sun blocks that may usually be used in cosmetics.

Examples of UVB screening agents that may be mentioned include:

(1) salicylic acid derivatives, in particular homomethyl salicylate and octyl salicylate;
(2) cinnamic acid derivatives, in particular the 2-ethylhexyl p-methoxycinnamate sold by the company Givaudan under the name Parsol MCX;
(3) liquid β,β′-diphenylacrylate derivatives, in particular 2-ethylhexyl α-cyano-α,β′-diphenylacrylate, or octocrylene, sold by the company BASF under the name Uvinul N539;
(4) p-aminobenzoic acid derivatives;
(5) the 4-methylbenzylidenecamphor sold by the company Merck under the name Eusolex 6300;
(6) the 2-phenylbenzimidazole-5-sulfonic acid sold under the name Eusolex 232 by the company Merck;
(7) 1,3,5-triazine derivatives, in particular:
2,4,6-tris[p-(2′-ethylhexyl-1′-oxycarbonyl)anilino]-1,3,5-triazine sold by the company BASF under the name Uvinul T150, and
the dioctylbutamidotriazone sold by the company Sigma 3V under the name Uvasorb HEB;
   (8) mixtures of these screening agents.

Examples of UVA screening agents that may be mentioned include:

(1) dibenzoylmethane derivatives, in particular the 4-(tert-butyl)-4′-methoxydibenzoylmethane sold by the company Givaudan under the name Parsol 1789;
(2) benzene-1,4-bis(3-methylidenecamphor-10-sulfonic acid), optionally in partially or totally neutralized form, sold under the name Mexoryl SX by the company Chimex;
(3) benzophenone derivatives, for example:
2,4-dihydroxybenzophenone (benzophenone-1);
2,2′,4,4′-tetrahydroxybenzophenone (benzophenone-2);
2-hydroxy-4-methoxybenzophenone (benzophenone-3), sold under the name Uvinul M40 by the company BASF;
2-hydroxy-4-methoxybenzophenone-5-sulfonic acid (benzophenone-4), and also its sulfonate form (benzophenone-5), sold by the company BASF under the name Uvinul MS40;
2,2′-dihydroxy-4,4′-dimethoxybenzophenone (benzophenone-6);
5-chloro-2-hydroxybenzophenone (benzophenone-7);
2,2′-dihydroxy-4-methoxybenzophenone (benzophenone-8);
the disodium salt of 2,2′-dihydroxy-4,4′-dimethoxybenzophenone-5,5′-disulfonic acid (benzophenone-9);
2-hydroxy-4-methoxy-4′-methylbenzophenone (benzophenone-10);
benzophenone-11;
2-hydroxy-4-(octyloxy)benzophenone (benzophenone-12);
   (4) silane derivatives or polyorganosiloxanes comprising a benzophenone group;
   (5) anthranilates, in particular the menthylanthranilate sold by the company Haarman & Reimer under the name Neo Heliopan MA;
   (6) compounds comprising, per molecule, at least two benzazolyl groups or at least one benzodiazolyl group, in particular 1,4-bis(benzimidazolyl)phenylene-3,3′5,5′-tetrasulfonic acid, and also its salts, sold by the company Haarman & Reimer;
   (7) silicon derivatives of N-substituted benzimidazolyl-benzazoles or of benzofuranyl-benzazoles, and in particular:
2-[1-[3-[1,3,3,3-tetramethyl-1-[(trimethylsilyl)oxy]-disiloxanyl]propyl]-1H-benzimidazol-2-yl]benzoxazole;
2-[1-[3-[1,3,3,3-tetramethyl-1-[(trimethylsilyl)oxy]-disiloxanyl]propyl]-1H-benzimidazol-2-yl]benzothiazole;
2-[1-(3-trimethylsilanylpropyl)-1H-benzimidazol-2-yl]benzoxazole;
6-methoxy-1,1′-bis(3-trimethylsilanylpropyl)-1H,1′H-[2,2′]bibenzimidazolylbenzoxazole;
2-[1-(3-trimethylsilanylpropyl)-1H-benzimidazol-2-yl]benzothiazole;
   which are described in document EP-A-1 028 120;
   (8) triazine derivatives, and in particular 2,4-bis{[4-(2-ethylhexyloxy)-2-hydroxyl]phenyl}-6-(4-methoxyphenyl)-1,3,5-triazine sold by the company Ciba Geigy under the name Tinosorb S, and 2,2′-methylenebis[6-(2H-benzotriazol-2-yl)-4-(1,1,3,3-tetramethylbutyl)phenol] sold by the company Ciba Geigy under the name Tinosorb M;
   (9) benzotriazole silicones, which are described especially in document EP-A-0 392 883, in particular the benzotriazole silicone of formula:

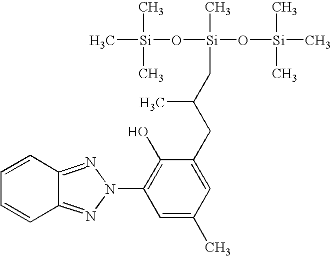

(10) mixtures thereof.

A mixture of several of these screening agents may also be used.

IX) Skin-colouring and hair-colouring agents, in particular skin-colouring agents such as dihydroxyacetone (DHA), natural dyes such as plant extracts, for instance extracts of sorghum, and optical brighteners.

Optical brighteners are a family of fluorescent substances that are well known to those skilled in the art. Such compounds are described in “Fluorescent Whitening Agent, Encyclopedia of Chemical Technology, Kirk-Othmer”, vol. 11, pp. 227-241, 4th edition, 1994, Wiley. They are agents that optically bleach the skin, consisting of chemical compounds endowed with fluorescence properties, which absorb in the ultraviolet range (maximum absorption at a wavelength of less than 400 nm) and re-emit the energy via fluorescence at a wavelength of between 380 nm and 830 nm. An emission of energy of between 400 nm and 480 nm results in an emission in the blue region of the visible range, which contributes, when this emission takes place on the skin, towards visually whitening it.

They may be defined more particularly as compounds that absorb essentially in the UVA and UVB range between 290 and 400 nm and re-emit essentially between 400 and 525 nm. Among the optical brighteners that are preferably used are stilbene derivatives, coumarin derivatives, oxazole and benzoxazole derivatives and imidazole derivatives. Such compounds are readily commercially available. The optical brighteners preferably used in the product according to the invention are the distyryl-4,4′-biphenyl disulfonate sold by the company Ciba Geigy under the name Tinopal CBS-X®. Examples that may also be mentioned include sodium 4,4′-bis[(4,6-dianilino-1,3,5-triazin-2-yl)amino]stilbene-2,2′-disulfonate and 2,5-thiophenediylbis(5-tert-butyl-1,3-benzoxazole), sold by the company Ciba Geigy under the names Tinopal SOP® and Uvitex OB®.

X) Concealing active agents, such as vitamin K1 and its derivatives, and coumarins.
XI) Antiperspirant active agents such as aluminium and/or zirconium salts, for instance aluminium chlorohydrate, aluminium chlorohydrex, aluminium chlorohydrex PEG, aluminium chlorohydrex PG, aluminium dichlorohydrate, aluminium dichlorohydrex PEG, aluminium dichlorohydrex PG, aluminium sesquichlorohydrate, aluminium sesquichlorohydrex PEG, aluminium sesquichlorohydrex PG, alun salts, aluminium sulfate, aluminium zirconium octachlorohydrate, aluminium zirconium pentachlorohydrate, aluminium zirconium tetrachlorohydrate, aluminium zirconium trichlorohydrate and more particularly the aluminium chlorohydrate sold by the company Reheis under the name Reach 301 or 303, or by the company Guilini Chemie under the name Aloxicoll PF 40, and the aluminium zirconium salt sold by the company Reheis under the name Reach AZP-908-SUF; complexes of zirconium hydroxychloride and of aluminium hydroxychloride with an amino acid, such as those described in document U.S. Pat. No. 3,792,068, which are commonly known as “ZAG complexes”, for instance aluminium zirconium octachlorohydrex GLY, aluminium zirconium pentachlorohydrex GLY, aluminium zirconium tetrachlorohydrate GLY and aluminium zirconium trichlorohydrate-GLY.
XII) Deodorant active agents, such as zinc pyrrolidonecarboxylate (more commonly known as zinc pidolate), zinc sulfate, zinc chloride, zinc lactate, zinc gluconate and zinc phenolsulfonate, 2,4,4′-trichloro-2′-hydroxydiphenyl ether (Triclosan), 2,4-dichloro-2′-hydroxydiphenyl ether, 3′,4′,5′-trichlorosalicylanilide, 1-(3′4′,-dichlorophenyl)-3-(4′-chlorophenyl)urea (Triclocarban) or 3,7,11-trimethyldodeca-2,5,10-trienol (Farnesol); quaternary ammonium salts, for instance cetyltrimethylammonium salts and cetylpyridinium salts; chlorhexidine and its salts; diglyceryl monocaprate, diglyceryl monolaurate and glyceryl monolaurate; polyhexamethylene biguanide salts.
XIII) Hair-treating active agents such as (1) hair-loss inhibitors and hair-growth stimulators, such as minoxidil, biotin, aminexil, cysteine, finasteride, 2,4-dipyrimidine N-oxide, panthenol and derivatives, flavanone T, calcium antagonists, for instance diltiazem, verapamil, alverine and nifedipine, hormones, for instance progesterone, FP receptor agonists, for instance latanoprost, type-1 15-hydroxyprostaglandin dehydrogenase inhibitors, prostaglandins and derivatives thereof, or more generally any plant extract having type I or II anti-5-alpha reductase activity; (2) antidandruff agents such as zinc pyrithione, 1-hydroxy-2-pyrrolidone derivatives or selenium sulfides.
XIV) Hair-removing agents, which are used to inhibit the growth of hairs, such as thioglycolic acid and its derivatives; dithioglycolic acid and its derivatives; the serine proteases described in document U.S. Pat. No. 6,407,056; caffeic acid; quercetin; propyl gallate; norhydroguaiaretic acid or NDGA; indomethacin; eflornithine hydrochloride; plant extracts as described in document U.S. Pat. No. 6,171,595, for instance extracts of clove, of rosebud, of burnet or of Bengal gambir; the compounds described in document U.S. Pat. No. 6,075,052; tetramisole; sodium orthovanadate; levamisole; disodium chromoglycate; vanadium nitrate and gallium nitrate as described in document U.S. Pat. No. 6,020,006; the compounds described in documents U.S. Pat. Nos. 4,885,289, 4,720,489, 5,132,293, 5,096,911, 5,095,007, 5,143,925, 5,328,686, 5,440,090, 5,364,885, 5,411,991, 5,648,394, 5,468,476, 5,475,763, 5,455,608, 5,674,477, 5,728,736 and 5,652,273, WO-A-94/27586, WO-A-94/27563 and WO-A-98/03149; extracts of juniper as described in document U.S. Pat. No. 6,375,948.
XV) Pigments, which are used especially when the product obtained is intended for making up the skin, or when they are metal oxide pigments, when the product obtained is intended to constitute an antisun product for protecting coloured hair or skin from sunlight.

These pigments may be mineral and/or organic, interference, goniochromatic, fluorescent, white, coloured, nacreous or reflective, or in the form of flakes. The term “pigment” should be understood as meaning particles that are insoluble in the physiological medium of the composition.

Among the mineral pigments that may be mentioned are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue. Among the organic pigments that may be mentioned are carbon black, pigments such as organic lakes of barium, strontium, calcium or aluminium including those submitted for certification by the Food and Drug Administration (FDA) (for example D&C or FD&C) and those exempt from FDA certification, for instance lakes based on cochineal carmine.

The nacreous pigments or nacres may be chosen from white nacreous pigments such as mica coated with titanium or with bismuth oxychloride, coloured nacreous pigments such as titanium mica with iron oxides, titanium mica with, especially, ferric blue or chromium oxide, titanium mica with an organic pigment of the abovementioned type, and nacreous pigments based on bismuth oxychloride. Among the commercially available nacres that may be mentioned are the nacres sold under the names Timica® and Flamenco® by the company Engelhard and the nacres sold under the name Timiron® by the company Merck.

It is also possible to use goniochromatic pigments, for instance pigments with a multilayer interference structure, for example of Al/SiO₂/Al/SiO₂/Al structure, sold by the company Dupont de Nemours; of Cr/MgF₂/Al/MgF₂/Cr structure sold under the name Chromaflair® by the company Flex; of MOS₂/SiO₂/Al/SiO₂/MOS₂, Fe₂O₃/SiO₂/Al/SiO₂/Fe₂O₃ or Fe₂O₃/SiO₂/Fe₂O₃/SiO₂/Fe₂O₃ structure sold under the name Sicopearl® by the company BASF; of MOS₂/SiO₂/mica-oxide/SiO₂/MoS₂, Fe₂O₃/SiO₂/mica-oxide/SiO₂/Fe₂O₃, TiO₂/SiO₂/TiO₂ or TiO₂/Al₂O₃/TiO₂ structure, sold under the name Xirona® by the company Merck. The pigments sold under the name Infinite Colors® from the company Shiseido may also be mentioned.

It is also possible to use reflective pigments, for instance particles with a silver-coated glass substrate, in the form of platelets, such as those sold, for example, under the name Microglass Metashine REFSX 2025 PS® by the company Toyal; particles with a glass substrate coated with nickel/chromium/molybdenum alloy, such as those sold, for example, under the names Crystal Star GF 55® and GF 2525® by the company Toyal; pigments of brand name Reflecks®, sold by the company Engelhard, comprising a glass substrate coated with brown iron oxide; particles comprising a stack of at least two polymer layers, for instance those sold by the company 3M under the name Mirror Glitter®.

Examples of liquid-crystal goniochromatic particles that may be used include those sold by the company Chemx and also the product sold under the name Helicone® HC by the company Wacker.

XVI) Dyes, which give a colour to the film(s) and/or to the aqueous composition, especially water-soluble dyes such as copper sulfate, iron sulfate, water-soluble sulfopolyesters, rhodamines, natural dyes, for instance carotene and beetroot juice, methylene blue, caramel, the disodium salt of tartrazine and the disodium salt of fuschin, and mixtures thereof. Liposoluble dyes may also optionally be used. The dye(s) is (are) preferably present in the film(s). The aqueous composition may thus be coloured at the time of use according to the shade desired at that time.

The dyes and pigments allow the colour to be modified as a function of the desired aim (for example healthy complexion or concealing effect).

XVII) Fragrances, which may be of any type, either composite fragrances comprising a mixture of odoriferous materials, or an isolated odoriferous material. Thus, the kit may comprise several films each comprising a different odoriferous material such that several films mixed into the aqueous composition give a particular fragrance. Fragrances with muscle-relaxing properties, which afford a relaxing effect when the product is applied to the skin, may also be introduced.

The odoriferous materials are compounds usually used by perfumers and they are described especially in S. Arctander, Perfume and Flavor Chemicals (Montclair, N.J., 1969) and S. Arctander, Perfume and Flavor Materials of Natural Origin (Elizabeth, N.J., 1960) and in “Flavor and Fragrance Materials—1991”, Allured Publishing Co. Wheaton, Ill. USA.

They may be natural products (essential oils, absolutes, resinoids, resins or concretes) and/or synthetic products (hydrocarbons, alcohols, aldehydes, ketones, ethers, acids, esters, acetals, ketals or nitrites, which may be saturated or unsaturated, and aliphatic or cyclic).

Examples of essential oils comprise essential oils of lemon, of orange, of aniseed, of bergamot, of rose, of geranium, of ginger, of neroli, of basil, of rosemary, of cardamom, of camphor, of cedar, of camomile, of sandalwood and of sage, and mixtures thereof, without this list being limiting.

Examples of other odoriferous materials are especially geraniol, geranyl acetate, farnesol, borneol, bornyl acetate, linalol, linalyl acetate, linalyl propionate, linalyl butyrate, tetrahydrolinalol, citronellol, citronellyl acetate, citronellyl formate, citronellyl propionate, dihydromyrcenol, dihydromyrcenyl acetate, tetrahydromyrcenol, terpineol, terpinyl acetate, nopol, nopyl acetate, nerol, neryl acetate, 2-phenylethanol, 2-phenylethyl acetate, benzyl alcohol, benzyl acetate, benzyl salicylate, styrallyl acetate, benzyl benzoate, amyl salicylate, dimethylbenzylcarbinol, trichloromethylphenylcarbinyl acetate, p-tert-butylcyclohexyl acetate, isononyl acetate, vetiveryl acetate, vetiverol, α-hexylcinnamaldehyde, 2-methyl-3-(p-tert-butylphenyl)propanol, 2-methyl-3-(p-isopropylphenyl)propanal, 3-(p-tert-butylphenyl)propanal, 2,4-dimethylcyclohex-3-enylcarboxaldehyde, tricyclodecenyl acetate, tricyclodecenyl propionate, 4-(4-hydroxy-4-methylpentyl)-3-cyclohexenecarboxaldehyde, 4-(4-methyl-3-pentenyl)-3-cyclohexenecarboxaldehyde, 4-acetoxy-3-pentyltetrahydropyran, 3-carboxymethyl-2-pentylcyclopentane, 2-n-heptylcyclopentanone, 3-methyl-2-pentyl-2-cyclopentenone, menthone, carvone, tagetone, geranyl acetone, n-decanal, n-dodecanal, 9-decen-1-ol, phenoxyethyl isobutyrate, phenylacetaldehyde dimethyl acetal, phenylacetaldehyde diethyl acetal, geranonitrile, citronellonitrile, cedryl acetate, 3-isocamphylcyclohexanol, cedryl methyl ether, isolongifolanone, aubepinonitrile, aubepine, heliotropine, coumarin, eugenol, vanillin, diphenyl ether, citral, citronellal, hydroxycitronellal, damascone, ionones, methylionones, isomethylionones, solanone, irones, cis-3-hexenol and its esters, indane musks, tetralin musks, isochroman musks, macrocyclic ketones, macrolactone musks and ethylene brassylate, and mixtures thereof.

XVIII) Polymers which, as indicated above, may be those intrinsically present in the anhydrous film(s), but it is also possible to use other polymers present in anhydrous films and liable to afford particular rheological properties to the aqueous composition. Examples of polymers that may be mentioned include modified or unmodified carboxyvinyl polymers, such as the products sold under the names Carbopol (INCI name: carbomer) and Pemulen (INCI name: Acrylates/C10-30 alkyl acrylate crosspolymer) by the company Noveon; polyacrylates and polymethacrylates such as the products sold under the names Lubrajel and Norgel by the company Guardian or under the name Hispagel by the company Hispano Chimica; polyacrylamides; 2-acrylamido-2-methylpropanesulfonic acid polymers and copolymers, which are optionally crosslinked and/or neutralized, for instance the poly(2-acrylamido-2-methylpropane-sulfonic acid) sold by the company Clariant under the name “Hostacerin AMPS” (INCI name: ammonium polyacryldimethyltauramide); crosslinked anionic copolymers of acrylamide and of AMPS, which are in the form of a W/O emulsion, such as those sold under the name Sepigel 305 (INCI name: Polyacrylamide/C13-14 Isoparaffin/Laureth-7) and under the name Simulgel 600 (INCI name: Acrylamide/Sodium acryloyldimethyltaurate copolymer/Isohexadecane/Polysorbate 80) by the company SEPPIC; polysaccharide biopolymers, for instance xanthan gum, guar gum, carob gum, acacia gum, scleroglucans, chitin and chitosan derivatives, carrageenans, gellans, alginates, celluloses such as microcrystalline cellulose, carboxymethylcellulose, hydroxymethylcellulose and hydroxypropylcellulose; associative polymers, for instance associative polyurethanes, copolymers comprising at least two hydrocarbon-based lipophilic chains comprising from 6 to 30 carbon atoms, separated with a hydrophilic sequence, such as the polyurethanes sold under the names Serad FX1010, Serad FX1100 and Serad FX1035 by the company Huls (INCI name: Polyurethane), those sold under the names Rheolate 255, Rheolate 278 and Rheolate 244 by the company Rheox (INCI name: Polyether-urea-polyurethane), those sold under the names DW 1206F, DW 1206J, DW 1206B and DW 1206G by the company Röhm & Haas (INCI name: Polyurethane), and the product sold under the name Acrysol RM 2020 from the company Röhm & Haas. Fixing polymers such as those conventionally used in lakes and hairstyling products may also be used.
XIX) Electrolytes, and especially mono-, di- or trivalent metal salts and more particularly the salts of alkaline-earth metals and in particular barium, calcium and strontium salts, alkali metal salts, for example sodium and potassium salts, and also magnesium, beryllium, yttrium, lanthanum, cerium, praseodymium, neodymium, promethium, samarium, europium, gadolinium, terbium, dysprosium, holmium, erbium, thulium, ytterbium, lutetium, lithium, tin, zinc, manganese, cobalt, nickel, iron, copper, rubidium, aluminium, silicon and selenium salts, and mixtures thereof.

The ions constituting these salts may be chosen, for example, from carbonates, bicarbonates, sulfates, phosphates, sulfonates, glycerophosphates, borates, bromides, chlorides, nitrates, acetates, hydroxides and persulfates, and also α-hydroxy acid ions (citrates, tartrates, lactates and malates) or fruit acid ions, β-hydroxy acid ions (salicylates, 2-hydroxy alkanoates, n-alkylsalicylates and n-alkanoylsalicylates), or amino acid ions (aspartate, arginate, glycocholate and fumarate).

It is also possible to use a mixture of these salts, including natural mixtures or mixtures whose composition is close to that of a natural mixture, and in particular of an aqueous mixture comprising from 30% to 35% magnesium chloride, from 20% to 28% potassium chloride, from 3% to 10% sodium chloride, from 0.2% to 1% calcium chloride, from 0.1% to 0.6% magnesium bromide and from 0.1% to 0.5% of insoluble matter, the mixture being known as “Dead Sea Bath Salts” since it corresponds to the main salts contained in the Dead Sea.

Preferred electrolytes are especially sodium chloride, magnesium chloride and Dead Sea salts.

XX) pH adjusters and especially those usually used in cosmetics to adjust the pH of compositions to the desired value. They may be acids or bases chosen from mineral bases, for instance sodium hydroxide, organic bases, for instance amines (for example triethanolamine), mineral acids, for instance hydrochloric acid, and organic acids, for instance citric acid.
XXI) Preserving agents and especially any preserving agent usually used in cosmetics, in particular phenoxyethanol, para-hydroxybenzoic acid esters, also known as Parabens, for instance methyl para-hydroxybenzoate (methylparaben), ethyl para-hydroxybenzoate (ethylparaben) and propyl para-hydroxybenzoate (propylparaben) and mixtures thereof; formaldehyde-releasing agents, for instance imidazolidinylurea or diazolidinylurea; haloalkynyl carbamates, for instance 3-iodo-2-propynyl butyl carbamate (IPBC); caprylyl glycol, also known as 1,2-octanediol; sodium benzoate; N-(3-chloroallyl)-hexaminium chloride (or Quaternium-15); polyhexamethylene biguanide hydrochloride (CTFA name: polyaminopropyl biguanide); alkyltrimethylammonium bromides, for instance dodecyltrimethylammonium bromide, myristyltrimethylammonium bromide and hexadecyltrimethylammonium bromide, and mixtures thereof. These preserving agents may especially be in the aqueous composition.

The compounds mentioned above may be present in the final product in the usual amounts in the field under consideration, these amounts depending on the compound used and the desired aim. The compounds may be present, for example, in an amount ranging from 0.001% to 20% by weight and better still from 0.01% to 10% by weight relative to the weight of final product. According to the desired aim and the compound used, the compounds are present in one or more films or in the aqueous composition.

According to one particular embodiment of the invention, at least one film contains at least one compound chosen from compounds that are sensitive to an external stimulus, keratolytic agents, fillers and tensioning agents, and mixtures thereof.

The kit according to the invention may be used especially to obtain products intended to be applied to the skin, mucous membranes, the integuments or the hair, especially such as skincare products or as skin makeup products or as hair products or as antisun products.

The examples below of compositions according to the invention are given as non-limiting illustrations. The amounts therein are given as weight percentages, unless otherwise mentioned, and the names are given as the chemical name or as the CTFA name, depending on the case.

### Example 1

### Kit for an Antiwrinkle Product

Composition for film (initial mixture)

| | |
|---|---|
| Hydroxypropymethylcellulose | 10% |
| Glycerol | 5% |
| Panthenol | 2% |
| Adenosine | 0.15% |
| Purified water | 50% |

Procedure for preparing the film: the ingredients were all mixed together. The mixture was applied to a silicone paper using standard roller or doctor-blade coating techniques, to a thickness of about 500 μm. After drying in a tunnel with circulating hot air at a temperature of about 50° C., the film obtained was cut into the desired shape, for example into the form of square plates with a side length of 25 mm.

Aqueous composition (serum)

| Phase A | |
|---|---|
| Xanthan gum | 0.2% |
| Methyl paraben | 0.2% |
| Phenoxyethanol | 0.35 |
| Water | qs 100% |
| Phase B | |
| Triethanolamine | 0.2% |
| Polyacrylamide/C13-C14 isoparaffin/laureth-7 | 1% |
| (Sepigel 305) | |
| Diazolidinylurea | 0.3% |
| Glycerol | 7% |

Procedure for preparing the serum: phase A was heated to about 75° C., with stirring, and phase B was then added to phase A. Next, the heating was stopped while continuing the stirring, until the mixture cooled to room temperature. Gentle stirring was then continued for 30 minutes.

During use, the consumer mixes in the palm of her hand, with the fingers, one or more plates of anhydrous film with a dose of between 100 and 500 mg of the aqueous composition for about ten seconds. She gently massages the area to be treated so as to promote the spreading of the composition thus obtained onto the skin and the penetration of active agents it contains into the skin. The composition obtained may be applied to the face or the body, especially to the face for treating the wrinkles and fine lines of crowsfeet.

### Example 2

### Kit for an Anti-ageing Product

Anhydrous film

| | |
|---|---|
| Polyvinylpyrrolidone | 83% |
| Ascorbic acid | 7% |
| PEG-400 | 10% |

The film may be prepared in a fashion like that in Example 1, beginning with an initial aqueous mixture.

Aqueous composition (O/W emulsion)

| Phase A (aqueous phase) | |
|---|---|
| Xanthan gum | 0.25% |
| Preserving agent | 0.3% |
| Water | qs 100% |
| Phase B (oily phase) | |
| Shea butter | 2% |
| Methylglucose sesquistearate | 2% |
| PEG-20 methylglucose sesquistearate | 2% |
| Apricot kernel oil | 6% |
| Stearyl fatty alcohol | 2% |
| Phase C | |
| Polyacrylamine/C13-C14 isoparaffin/laureth-7 | 1% |
| (Sepigel 305) | |
| Preserving agent | 0.35% |
| Volatile silicone oil | 10% |

Procedure: phase A is heated to about 75° C., with stirring, and phase B is added to phase A with very vigorous stirring. Next, the heating is stopped while continuing the stirring, until the mixture has returned to room temperature. Phase C is then added. Gentle stirring is then continued for 30 minutes.

During use, the consumer mixes in the palm of her hand, with the fingers, one or more plates of anhydrous film with a dose of between 100 and 500 mg of the aqueous composition, for about 10 seconds. She gently massages the area to be treated so as to promote the spreading of the composition thus obtained onto the skin, and the penetration of the active agents it contains into the skin. The composition obtained may be applied to the face or the body, especially for preventing or treating the signs of ageing.

### Example 3

### Kit of Product for Greasy Skin

Anhydrous film

| | |
|---|---|
| Hydroxypropylmethylcellulose | 68% |
| Glycerol | 16% |
| Silica (Sunsphere H33 from the company Ashai Glass) | 16% |

The film may be prepared in a fashion like that in Example 1, beginning with an initial aqueous mixture.

Aqueous composition (O/W emulsion)

| Phase A (oily phase) | |
|---|---|
| Glyceryl stearate | 1.35% |
| Dimyristyl tartrate/cetearyl alcohol/12-15 | 2% |
| Pareth 7/PEG-25 laureth 25 (Cosmacol PSE from | |
| the company Sasol) | |
| PEG-400 stearate | 2.7% |
| Isohexadecane | 2% |
| Stearyl alcohol | 1.3% |
| Vitamin E | 0.1% |
| Preserving agent | 0.6% |
| Phase B (aqueous phase) | |
| Water | qs 100% |
| Potassium hydroxide | 1.5% |
| Preserving agent | 0.25% |
| Ammonium polyacryloyldimethyltaurate | 0.5% |
| (Hostacerin AMPS) | |
| Phase C | |
| Volatile silicone oil | 6% |

Procedure: phase A and phase B are separately heated to about 75° C., with stirring, and phase A is added to phase B with very vigorous stirring. Next, the heating is stopped while continuing the stirring, until the mixture has cooled to room temperature. Phase C is added. Gentle stirring is then continued for 30 minutes.

During use, the consumer mixes in the palm of her hand, with the fingers, one or more plates of anhydrous films with a dose of between 100 and 500 mg of the aqueous composition, for about 10 seconds. She gently massages the area to be treated so as to promote the spreading of the composition thus obtained onto the skin, and the penetration of the active agents it contains into the skin. The composition obtained may especially be applied to the face, where it gives an immediate matting effect.

### Example 4

### Care Cream

Anhydrous film

| | |
|---|---|
| Hydroxypropylmethylcellulose | 60% |
| Glycerol | 30% |
| Propylene glycol | 10% |

The film may be prepared in a fashion like that in Example 1, beginning with an initial aqueous mixture.

Aqueous composition:

| Phase A | |
|---|---|
| Xanthan gum | 0.2% |
| Methyl paraben | 0.2% |
| Phenoxyethanol | 0.35% |
| Water | qs 100% |
| Phase B | |
| Triethanolamine | 0.2% |
| Polyacrylamide/C13-C14 isoparaffin/laureth-7 | 1% |
| (Sepigel 305) | |
| Diazolidinylurea | 0.3% |
| Glycerol | 7% |

Procedure: phase A is heated to about 75° C., with stirring, and phase B is added to phase A. Next, the heating is stopped, while continuing the stirring until the mixture has cooled to room temperature. Gentle stirring is then continued for 30 minutes.

During use, the consumer mixes in the palm of her hand, with the fingers, one or more plates of anhydrous film with a dose of between 100 and 500 mg of the aqueous composition for about 10 seconds. She gently massages the area to be treated so as to promote the spreading of the composition thus obtained onto the skin. The composition obtained is a thick cream that has very good application properties.

This cream may be applied to the face or the body.

### Example 5

### Massage Cream

Anhydrous film

| | |
|---|---|
| Hydroxypropylmethylcellulose | 40% |
| Serad FX1100 | 20% |
| Glycerol | 30% |
| Propylene glycol | 10% |

The film may be prepared in a fashion like that in Example 1, beginning with an initial aqueous mixture.

Aqueous composition:

| Phase A | |
|---|---|
| Xanthan gum | 0.2% |
| Methyl paraben | 0.2% |
| Phenoxyethanol | 0.35% |
| Water | qs 100% |
| Phase B | |
| Triethanolamine | 0.2% |
| Polyacrylamide/C13-C14 isoparaffin/laureth-7 | 1% |
| (Sepigel 305) | |
| Glycerol | 7% |

Procedure: phase A is heated to about 75° C., with stirring, and phase B is added to phase A. Next, the heating is stopped, while continuing the stirring until the mixture has cooled to room temperature. Gentle stirring is then continued for 30 minutes.

During use, the consumer mixes in the palm of her hand, with the fingers, one or more plates of anhydrous film with a dose of between 100 and 500 mg of the aqueous composition for about 10 seconds. She gently massages the area to be treated so as to promote the spreading of the composition thus obtained onto the skin. The composition obtained is a cream that has thickening rheological characteristics associated with the polymer Serad FX. The advantage is the control of the amounts applied and of the massage qualities.

This cream may be applied to the face or to the body for a relaxing massage.

The above written description of the invention provides a manner and process of making and using it such that any person skilled in this art is enabled to make and use the same, this enablement being provided in particular for the subject matter of the appended claims, which make up a part of the original description and including the following four embodiments:

1. Kit for formulating a cosmetic product, comprising (1) an aqueous composition comprising at least one fatty substance and (2) at least one water-soluble anhydrous film comprising at least one compound that is capable of modifying the aqueous composition.

2. Kit for formulating a cosmetic product, comprising:

i) an aqueous composition; and

ii) at least one water-soluble anhydrous film comprising at least one compound which, for reasons of stability, can only be mixed with the composition extemporaneously in order to form the cosmetic product.

3. Kit for formulating a cosmetic product, comprising:
i) an aqueous composition;
ii) at least one first water-soluble anhydrous film; and
iii) at least one second water-soluble anhydrous film, the second film being different from the first, with regard to the concentration and/or the nature of at least one compound it contains.

4. Kit for the customized formulation of a cosmetic product, comprising:

i) an aqueous composition;

ii) a plurality of identical or different water-soluble anhydrous films, intended to be mixed with the aqueous composition to form the cosmetic product, and
iii) instructions for custom-formulating the cosmetic product as a function of the number of identical or different water-soluble anhydrous films to be mixed with the composition.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1-16. (canceled)

17. A process for formulating a cosmetic product, which comprises:
a) determining at least one characteristic of the cosmetic product to be formulated, the characteristic being due to at least one compound present either in a plurality of identical water-soluble anhydrous films, or in a plurality of water-soluble anhydrous films that are different from each other; and
b) adding to an aqueous composition one or a plurality of the identical or different films, the number depending on the characteristic of the cosmetic product to be formulated.

18. The process of claim 17, wherein said process modifies the rheology, colour and/or fragrance characteristics of the aqueous composition.

19. The process of claim 17, wherein said films are formed from at least one water-soluble or water-dispersible polymer and comprise at least one compound chosen from keratolytic agents, tensioning agents, moisturizing active agents, anti-seborrhoeic agents, anti-ageing active agents, antimicrobial active agents, anti-inflammatory or calmative active agents, lipolytic or slimming active agents, fillers, sunscreens, skin-colouring or hair-colouring agents, concealing active agents, antiperspirant active agents, deodorant active agents, hair-treating active agents, hair-removing agents, pigments, dyes, polymers, fragrances, electrolytes, pH adjusters and preserving agents, and mixtures thereof.

20. The process of claim 17, wherein said aqueous composition is in the form of an emulsion comprising an aqueous phase and an oily phase dispersed in each other.

21. The process according to claim 17, comprising a plurality of wate-soluble anhydrous films, wherein the second film being different from the first, with regard to the concentration and/or the nature of at least one compound therein.

22. The process according to claim 17, wherein the film is formed from at least one water-soluble or water-dispersible polymer.

23. The process according to claim 22, wherein the polymer is chosen from polymers of protein type; chitin-based or chitosan-based polymers; polysaccharide polymers; acrylic polymers or copolymers; vinyl polymers; polymers of natural origin; and mixtures thereof.

24. The process according to claim 17, wherein the film has a thickness of from 10 μm to 1000 μm.

25. The process according to claim 17, wherein the film comprises less than 10% water.

26. The process according to claim 23, wherein the amount of polymer ranges from 5% to 100% by weight relative to the total weight of the film.

27. The kit according to claim 17, wherein the film further comprises one or more plasticizers.

28. The process according to claim 17, wherein the aqueous composition comprises water and at least one fatty substance.

29. The process according to claim 17, wherein the compound is chosen from cosmetic active agents and formulation adjuvants, and mixtures thereof.

30. The process according to claim 17, wherein the aqueous composition comprises water and one or more compounds chosen from cosmetic active agents and formulation adjuvants, and mixtures thereof.

31. The process according to claim 31, wherein the compound(s) are chosen from moisturizing active agents, anti-seborrhoeic agents, anti-ageing active agents, antimicrobial active agents, anti-inflammatory or calmative active agents, lipolytic or slimming active agents, fillers, sunscreens, skin-colouring or hair-colouring agents, concealing active agents, antiperspirant active agents, deodorant active agents, hair-treating active agents, hair-removing agents, pigments, dyes, polymers, fragrances, electrolytes, pH adjusters and preserving agents, and mixtures thereof.

32. The proecss according to claim 17, wherein at least one film comprises at least one compound selected from the group consisting of keratolytic agents, fillers, tensioning agents, and compounds that are sensitive to an external stimulus.
